(19) United States
(12) Patent Application Publication
(10) Pub. No.: US 2026/0283839 A1
(43) Pub. Date: Sep. 24, 2026

(54) Integrated Cold Plunge Therapy System with Hydro-Circulation, Ice- Forming Cooling, Multi-Wavelength Light Therapy, Sound Therapy, and Mobility Enhancement

(71) Applicants: Adam Fischer, San Diego, CA (US); Tyler Fish, San Diego, CA (US)

(72) Inventors: Adam Fischer, San Diego, CA (US); Tyler Fish, San Diego, CA (US)

(21) Appl. No.: 19/568,978

(22) Filed: Mar. 17, 2026

Related U.S. Application Data
(60) Provisional application No. 63/773,488, filed on Mar. 18, 2025.
(60) Provisional application No. 63/773,487, filed on Mar. 18, 2025.
(60) Provisional application No. 63/773,480, filed on Mar. 18, 2025.
(60) Provisional application No. 63/809,280, filed on May 20, 2025.
(60) Provisional application No. 63/818,623, filed on Jun. 5, 2025.

(51) Int. Cl. A61F 7/00; A61H 33/00

(52) U.S. Cl. CPC A61F 7/0053; A61F 7/0085; A61H 33/0087; A61F 2007/0056; A61F 2007/0093; A61H 2201/0242; A61H 2201/10; A61H 2201/5007; A61H 2201/5012; A61H 2203/02; A61H 2230/045; A61H 2230/065; A61H 2230/425; A61H 2230/505

(57) ABSTRACT

An integrated cold plunge therapy system comprising: a hydro-circulation subsystem with single or multiple pump configurations and directional nozzles; an ice-forming cooling mechanism using thermally conductive coils coupled to a closed-loop refrigeration circuit; a multi-wavelength light therapy subsystem including infrared, red, green, blue, and ultraviolet emitters in waterproof configurations; sound therapy subsystem with liquid-coupled transducers for acoustic stimulation and ice management; and a caster wheel mobility system available in integrated and detachable attachment embodiments. The system is operable via manual or digital controls. An intelligent controller incorporating machine learning or artificial intelligence receives pre-session and real-time biomarker data from wearable sensors and dynamically adjusts all subsystem parameters—including liquid temperature, ice formation rate, circulation intensity, light emission wavelength and power, and acoustic frequency—to deliver personalized, safe, and adaptive cold plunge therapy. The controller supports biometric authentication, voice interfaces, remote clinician access, cloud-based multi-device coordination, and safety override functions.

### CROSS-REFERENCE TO RELATED APPLICATIONS

This application claims the benefit of, priority to, and incorporates by reference the following U.S. Provisional Patent Applications: (i) 63/773,480, filed on Mar. 18, 2025; (ii) 63/773,487, filed on Mar. 18, 2025; (iii) 63/773,488, filed Mar. 18, 2025; (iv) 63/809,280 filed May 20, 2025; (v) 63/818,621, filed Jun. 5, 2025; and, (vi) 63/818,623, filed Jun. 6, 2025; all of which are incorporated herein in their entirety.

### FIELD OF THE INVENTION

The present invention relates generally to health maintenance and therapeutic hydrotherapy systems, and more specifically to an integrated cold plunge system comprising: (a) a hydro-circulation subsystem with multi-pump configurations; (b) an ice-forming cooling mechanism; (c) a multi-wavelength light therapy subsystem; (d) an optional sound therapy subsystem; and (e) a caster-wheel-based mobility system. The invention further pertains to both manual and intelligent, sensor-responsive control architectures for regulating liquid temperature, circulation, light therapy, and acoustic therapy parameters. The invention is applicable to residential, athletic recovery, and clinical environments, and encompasses both integrated and modular attachment embodiments.

### BACKGROUND OF THE INVENTION

Cold plunge therapy, wherein a user immerses their body in sub-ambient temperature liquid, is increasingly recognized for its benefits including muscle recovery, inflammation reduction, neuromuscular regulation, and metabolic stimulation. Conventional cold plunge systems are limited in their ability to achieve and sustain the lowest possible temperatures efficiently, to deliver targeted or full-body hydrodynamic stimulation, or to adapt therapy parameters in response to the physiological state of the user.

Hydro-jet and hydro-circulator systems for hot tubs and spa applications are known; however, systems specifically designed and engineered for the sub-freezing and near-freezing temperatures of cold plunge applications—including ice formation, antifreeze operating modes, and cold-resistant pump and nozzle materials—have not been adequately addressed in the prior art.

Ice-forming systems for cold plunge tubs utilizing thermally conductive coils to achieve temperatures below those achievable by liquid chillers alone, while integrating automatic de-icing modes and intelligent temperature feedback, represent a significant unmet need.

Photobiomodulation (light therapy) using red, near-infrared, blue, green, and ultraviolet wavelengths has demonstrated cellular regeneration, antimicrobial, anti-inflammatory, and nervous system benefits. While cryotherapy has historically been considered incompatible with concurrent infrared therapy, emerging research indicates that cold immersion can enhance the molecular and cellular effects of specific photobiomodulation wavelengths. No prior system combines multi-wavelength in-liquid light therapy with cold plunge immersion in an integrated, synergistic device.

Cold plunge tubs are heavy when filled and difficult to reposition. Existing portability solutions require heavy lifting and do not address weight distribution, locking stability, self-leveling on uneven surfaces, or modular retrofitting of existing tubs.

There is a need for a unified system addressing all of the above, further enhanced by an intelligent controller capable of receiving biomarker data, applying machine learning or AI algorithms, and dynamically personalizing therapy parameters across all subsystems.

### BRIEF SUMMARY OF THE INVENTION

The present invention discloses an integrated cold plunge therapy system combining a hydro-circulation subsystem, an ice-forming cooling mechanism, a multi-wavelength light therapy subsystem, an optional sound therapy subsystem, and a caster-wheel-based mobility system within a single tub-based platform. These subsystems are configured to operate synergistically to accelerate heat exchange, deliver targeted photobiomodulation and acoustic stimulation, and provide portability without sacrificing stability.

In some embodiments, the hydro-circulation subsystem comprises one or more pumps—which may be arranged in series, in parallel, or in series-parallel combinations—connected to directional circulator nozzles positioned along the inner surfaces of the tub. Circulation patterns may be macro (full-body) or micro (localized to a specific anatomical region or injury site), and the nozzle positions and flow rates may be manually or automatically adjusted.

In some embodiments, the ice-forming cooling mechanism utilizes thermally conductive coils, preferably copper, arranged along the interior walls and/or base of the tub and coupled to a closed-loop refrigeration circuit. This mechanism lowers liquid temperature beyond the capability of a liquid chiller alone and may further include an automatic de-icing mode to manage ice accumulation.

In some embodiments, the multi-wavelength light therapy subsystem comprises arrays of light-emitting elements configured to emit one or more therapeutic wavelengths including infrared (750 nm-1 mm), red (620-750 nm), green (500-570 nm), blue (410-480 nm), and ultraviolet (UVA, UVB, UVC) wavelengths. Emitters are waterproofed (IP68-rated or equivalent) and may be embedded in tub walls, mounted on transparent panels, or housed in drop-in waterproof enclosures.

In some embodiments, the sound therapy subsystem comprises liquid-coupled transducers configured to emit frequencies ranging from sub-audible (below 20 Hz) through audible (20 Hz-20 kHz) to ultrasonic (above 20 kHz), which may be used for therapeutic somatosensory vibration, neural entrainment, acoustic microconvection, and ice dislodgement.

In some embodiments, the mobility system comprises a caster wheel assembly affixed to or integrated into the base of the tub, including locking mechanisms, optional shock-absorbing suspension, self-leveling features, and non-marking treads. In some embodiments, the assembly is available in an integrated (factory-installed) embodiment and a detachable modular embodiment for retrofitting existing tubs.

In some embodiments, the system is operated via a user-controlled manual or digital interface that accepts direct user settings of liquid temperature, circulation parameters, light therapy parameters, and sound therapy parameters.

In some embodiments, an adaptive controller incorporating a processor executes algorithms—which may include machine learning (ML) and artificial intelligence (AI) techniques—that receive biomarker inputs measured prior to and/or during use, and dynamically adjust all subsystem parameters to achieve personalized therapeutic outcomes. In some embodiments, the controller may store user-specific data locally or remotely (e.g., cloud server), authenticate users via biometric or passcode mechanisms, accept voice or natural language inputs, communicate with remote healthcare providers, and provide safety override and alert functions.

### DEFINITIONS

For purposes of this disclosure, the following definitions apply:
Biomarker: A measurement of a characteristic of a body (human or otherwise) indicative of a biological state, condition, or process, including without limitation heart rate (HR), heart rate variability (HRV), blood pressure, galvanic skin response, CO2 output, retinal activity, body temperature, skin color, circulation, and swelling.
Biometric: A physical or behavioral trait used to identify individuals, including without limitation fingerprints, eye scans, voice recognition, and facial recognition.
Hydro-circulator (equivalently, circulator): An orifice, nozzle, or other liquid motion generator or ejector that may pass liquid or modify the speed or direction of liquid to create circulation, turbulence, laminar flow, counter flow, vortex flow, micro flow, or macro flow. Multiple circulators may work independently or in unison. The liquid may have dissolved or mixed solids.
Intake port: As used herein, an intake port is an opening through which liquid flows out of the tub and into the pump system, to be filtered, chilled, and returned to the tub through the nozzles. One or more intake ports may be used. An intake port may be directional, with its orientation affecting which region of the tub liquid is drawn from.
Nozzle (equivalently, circulator nozzle or discharge port): As used herein, a nozzle is an opening through which liquid is expelled from the pump system back into the tub interior. One or more nozzles may be used. Nozzles may be fixed or adjustable in direction to control the pattern, velocity, and target region of liquid flow within the tub.
Coils: As used herein, coils refers to refrigerant tubing that runs along surfaces and through which refrigerant circulates as part of the cooling system. Even straight pipe runs are referred to as coils herein because in some embodiments the refrigerant circuit forms a loop.
Cold-plunge (equivalently, cold plunge): a tub containing liquid configured to remove heat from an immersed body.
Actuator: A device that instantiates the setting of device operational parameters, including without limitation motor speed controllers, relays, valves, and potentiometers.
Sensor: A device that determines the value of a measured parameter.
Photobiomodulation: The use of specific wavelengths of electromagnetic radiation to induce biological effects at the cellular and molecular level. As used herein, references to a specific wavelength refer to the peak wavelength of emission of the optical source. Optical emitters are broadband and carry a range of wavelengths; the peak wavelength is used throughout this disclosure as the identifying characteristic of each emission band.

### BRIEF DESCRIPTION OF THE FIGURES

FIG. 1 is a schematic diagram of the hydro-circulation subsystem.

FIG. 2 is a schematic diagram of the ice-forming cooling subsystem.

FIG. 3 is a schematic diagram of the caster wheel mobility system.

FIG. 4 is a schematic diagram of the multi-wavelength light therapy subsystem.

FIG. 5 is a schematic diagram of the intelligent control architecture.

FIG. 6 is an illustration of the cold plunge therapy system in assembled form.

FIG. 7 is a block diagram showing how pre-measured biomarker information and real-time biomarker information can be used independently to control the cold plunge.

FIG. 8 is a block diagram showing how pre-measured biomarker information and real-time biomarker information can be used together to control the cold plunge.

### DETAILED DESCRIPTION OF THE INVENTION

### I. Tub Structure

The cold plunge tub is configured to retain liquid and is formed from a non-corrosive, structurally stable material capable of withstanding hydrostatic pressure and thermal fluctuation under operating conditions, including sub-freezing temperatures. Suitable materials include, without limitation, stainless steel, composite fiberglass-reinforced polymer, high-density polyethylene, wood, and medically safe thermoplastics. The interior of the tub preferably comprises a stainless steel surface. The exterior may be coated or clad with thermally insulating material.

In some embodiments, the tub defines a volumetric basin having a bottom, which may be contoured, and vertical or sloped sidewalls. The tub may include a perimeter flange or rim serving as a structural support for integrated controls or mounting of auxiliary equipment. The internal geometry may be contoured to accommodate ergonomic positioning, with regions of variable depth, recessed seating, foot wells, or lumbar support areas, integrally formed or modularly added. The base may include molded channels or recesses for embedding cooling coils or housing low-profile circulator nozzles for upward fluid discharge. The underside of the tub may include drainage outlets, pressure relief valves, and access ports for serviceability.

In some embodiments, the tub base may be mounted upon a rigid chassis or frame constructed from powder-coated steel, aluminum, or composite material, providing structural reinforcement and facilitating installation of compressors, pumps, and electrical components. Alternatively, the tub may be an inflatable pool-like structure with mechanical subsystems housed separately. The base frame may include vibration-dampening features. Internal routing for plumbing, electrical lines, and signal cables is sealed from the liquid chamber using embedded conduits and/or bulkhead fittings. The structural design accommodates both integrated and modular embodiments, permitting hardware such as control systems, cooling modules, or sensor arrays to be permanently housed within the chassis or configured as externally attachable units.

### II. Hydro-Circulation Subsystem

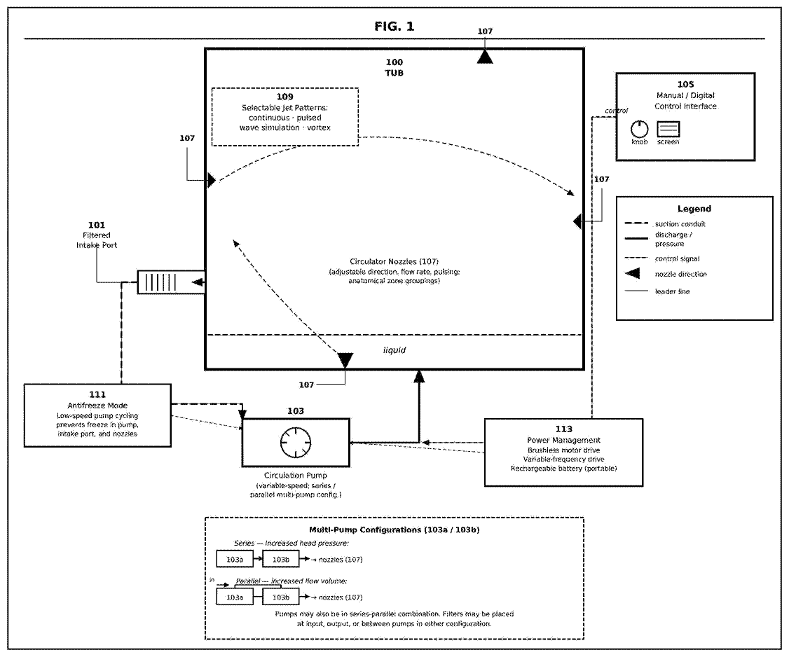

FIG. 1 is a schematic diagram of an embodiment of the hydro-circulation subsystem, illustrating the filtered intake (101), circulation pump (103), manual/digital flow control interface (105), adjustable directional nozzles (107), optional jet patterns (109), antifreeze operating mode (111), and power management system (113).

In some embodiments, the hydro-circulation subsystem is configured to create directed liquid circulation within the tub, promoting distribution of chilled liquid and delivering hydrodynamic stimulation to specific regions of the user's body. The combination of circulation and very cold liquid allows heat transfer to occur more rapidly than either alone.

In some embodiments, the hydro-circulation subsystem comprises a fluid intake port fitted with a debris filtration screen, one or more pumps, a network of fluid conduits (pipe, hose, or equivalent), and a plurality of circulator nozzles operatively coupled to the pump output. The pump may be of centrifugal, axial-circulation, or variable-speed impeller design. In one embodiment, circulation is effected by pumping air or gas into the liquid, or a gas-liquid combination.

In some embodiments, the pump is preferably driven by a brushless electric motor to reduce energy consumption and extend service life in low-temperature environments. The pump and associated control electronics are housed within a thermally insulated enclosure to prevent freezing or performance degradation during sub-freezing operation. The pump may be controlled via a manual control knob that provides incremental resistance points for precise flow adjustment, or via a digital interface. The control interface may include a wireless connectivity module enabling remote operation via a mobile application. In portable or off-grid embodiments, the pump may be powered by a rechargeable battery pack.

In some embodiments, in the attachment embodiment, the hydro-circulation unit may be affixed to an existing tub using a mounting bracket assembly, with a flexible inlet hose drawing liquid into the pump and an outlet nozzle directing pressurized liquid back into the tub. The system supports multiple user-selectable flow modes, including continuous high-flow mode, pulsed mode in which pressure varies at predetermined intervals, and wave simulation mode in which flow is cyclically modulated to produce varying therapeutic stimulation.

In some embodiments, the system employs multiple pumps. Pumps may be connected in series, in series with filters or other devices between them, or in parallel, or in combined series-parallel configurations, without limitation. A series configuration may increase head pressure at the output, thereby increasing flow rate in the tub. A parallel configuration may increase total liquid volume flow. Each pump may be motor-controlled or adjusted in output aperture independently to regulate pressure and flow characteristics. Filters and liquid cleaners may be placed at the input, output, or between pumps. In a parallel configuration, one pump may pass its output through a traditional particulate filter while another passes through an ozone and/or ultraviolet sanitation filter.

In some embodiments, the circulator nozzles may be positioned circumferentially around the tub wall to establish rotational circulation, or grouped into discrete anatomical zones (e.g., lower extremities, torso, cervical spine) for targeted therapy. Nozzles may be directional and independently adjustable in direction, flow rate, and pulsing pattern, with user-adjustable or controller-adjustable thrust levels. In some embodiments, intake ports may similarly be adjustable in direction, with their orientation affecting which region of the tub liquid is drawn from and thereby influencing overall circulation patterns within the tub. Nozzles may include internal circulation diverters or pulse-inducing components to modulate the output stream, producing pulsed pressure, oscillating flow, or vortex discharge.

In some embodiments, nozzles and intake orifices may be attached to hoses connected to a separate circulator pump and affixed to the tub interior without penetrating the tub surface, using suction, magnetic, or clamp attachment systems.

In some embodiments, there may be protected zones of the user's body that are wrapped or isolated to prevent cooling, fed by separate circulation nozzles delivering warmed or ambient-temperature liquid. Isolation may be achieved by blocking off a portion of the tub from the main liquid flow, by neoprene wraps, or by wraps incorporating inlet and outlet tubes through which warmed liquid is circulated to protect a localized injury site.

### III. Ice-Forming Cooling Subsystem

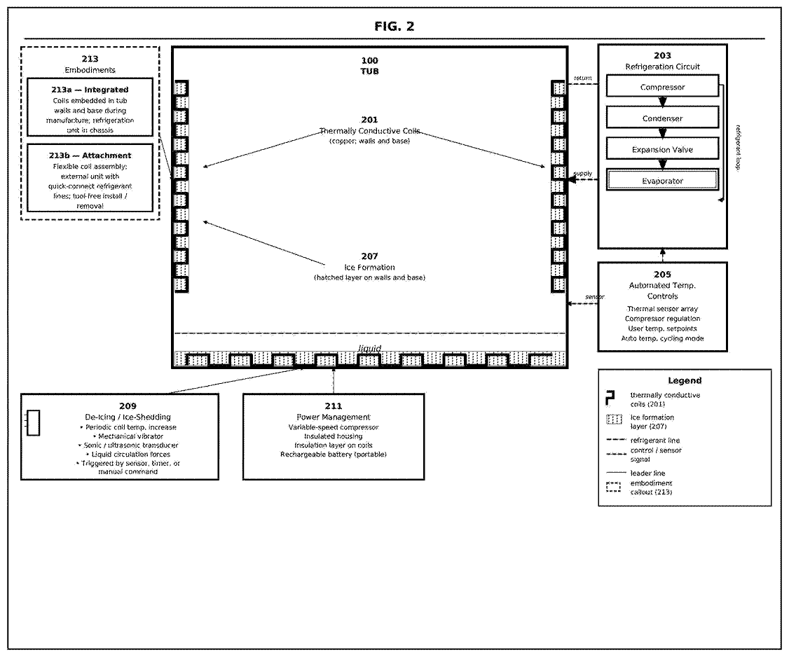

FIG. 2 is a schematic diagram of an embodiment of the ice-forming cooling subsystem, illustrating the thermally conductive coils (201), closed-loop refrigeration system (203), automated temperature controls (205), tub interior surfaces where ice formation occurs (207), automated de-icing mode (209), and energy-efficient power management (211), together with integrated and attachment embodiment configurations (213).

In some embodiments, the ice-forming cooling subsystem is configured to lower liquid temperature beyond the capability of a liquid chiller alone. In some embodiments, the liquid chiller first chills the liquid, chilling the liquid to a temperature above the freezing point of the liquid, and the ice-forming mechanism then supplies additional cooling beyond the capability of the chiller alone, forming ice within the tub and thereby circulating the liquid through a below-freezing thermal environment. This approach utilizes the latent heat of fusion of the liquid (or a component thereof) to absorb additional thermal energy. The ice may be the solid state of the liquid used in the tub, which is not necessarily water and may include dissolved solids.

In some embodiments, the cooling subsystem comprises a closed-loop refrigeration circuit including a compressor, a condenser, an expansion valve, and an evaporator, interconnected in a closed fluidic path. The compressor may be scroll, rotary, fixed-speed, or variable-speed. The refrigerant is selected to be environmentally compliant (e.g., R-134a, R-290, or other low global warming potential formulations).

In some embodiments, thermally conductive coils, preferably copper, are arranged in contact with the interior walls and/or base of the tub and serve as the primary thermal exchange interface between the refrigeration circuit and the tub liquid. The coils may be embedded within the structural walls or molded into a recess in the interior lining (integrated embodiment), or mounted externally and thermally coupled through the tub wall using conductive adhesive compounds or clamping brackets (attachment embodiment). In alternative embodiments, a thermal plate exchanger may be used, wherein tub liquid does not pass through an external chiller but is cooled by contact with the cold inner wall.

In some embodiments, in the attachment embodiment, the cooling unit is a modular device with a flexible copper coil assembly positioned against the tub interior surface and secured using an adjustable mounting system. The external cooling unit connects to the flexible coil assembly via quick-connect refrigerant lines, enabling tool-free installation and removal. The refrigeration system utilizes a variable-speed compressor to minimize power consumption while maintaining target temperatures. The cooling unit is enclosed within an insulated housing to reduce thermal losses, and an energy-saving insulation layer applied to the coils further enhances cooling efficiency. In portable embodiments, the cooling unit may be powered by a rechargeable battery system. The control system supports an automated temperature cycling mode in which cooling intensity is periodically adjusted to optimize energy efficiency and maintain consistent ice formation.

In some embodiments, in certain operational modes, the chilled coils further induce formation of a surface ice layer along the tub lining. This constitutes in-tub ice formation, in which ice is formed directly within the tub by the action of the coils on the tub walls or lining, as distinguished from an external ice-forming mechanism that forms ice outside the tub and introduces it into the tub liquid. The system includes an ice-shedding mechanism to distribute ice into the liquid. Ice-shedding may be implemented via a mechanical vibrator (linear or rotary actuator) operatively coupled to the tub wall or cooling coil assembly, a sonic or ultrasonic transducer, user movement, or liquid circulation forces. The controller may activate ice-shedding in response to sensed ice thickness, preprogrammed time intervals, or manual command.

In some embodiments, to prevent excessive ice buildup that could obstruct water flow or compromise tub functionality, the system incorporates an automatic de-icing mode that periodically raises the coil temperature slightly, managed by a thermal sensor array continuously monitoring water temperature and ice thickness.

In some embodiments, in a separated embodiment, the liquid chiller is a standalone unit with input and output hoses for cooling liquid and returning it to the tub. In another separated embodiment, the ice-forming mechanism is a standalone unit that accepts liquid from the tub, converts it to ice, and feeds ice back into the tub, with the controller managing ice formation rate to maintain target tub liquid level and temperature.

### IV. Multi-Wavelength Light Therapy Subsystem

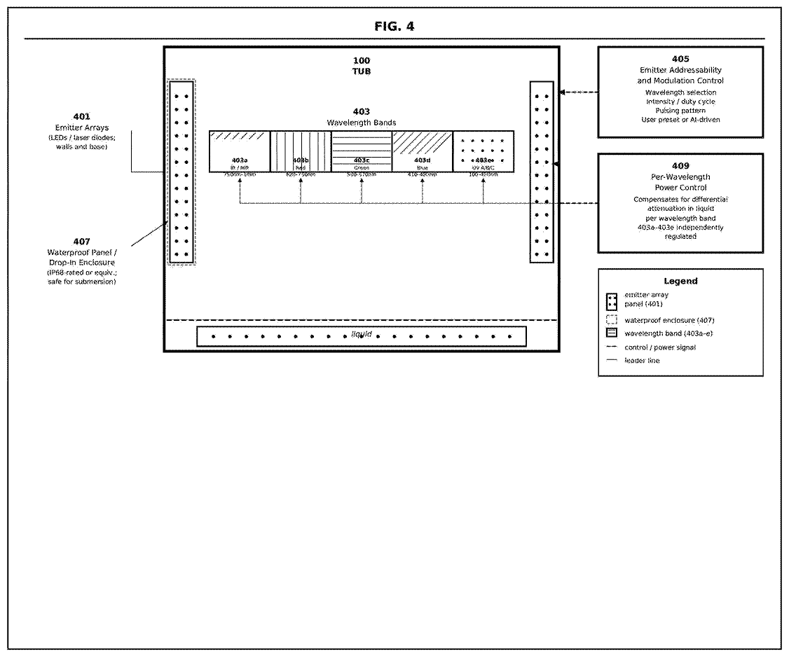

FIG. 4 is a schematic diagram of an embodiment of the multi-wavelength light therapy subsystem, illustrating emitter arrays (401) configured for one or more wavelength bands (403), individual emitter addressability and modulation control (405), waterproof panel or drop-in enclosure configurations (407), and power control per wavelength to compensate for liquid attenuation (409).

In some embodiments, the light therapy subsystem comprises a matrix of light-emitting elements—such as LEDs, laser diodes, or phosphor-converted emitters—configured to emit specific therapeutic wavelengths in the visible and near-infrared (NIR) spectrum. The system may include two or more wavelengths selected from: infrared (far, mid, or near infrared, approximately 750 nm-1 mm); red light (approximately 620-750 nm); green light (approximately 500-570 nm); blue light (approximately 410-480 nm); ultraviolet A (315-400 nm); ultraviolet B (280-315 nm); and ultraviolet C (100-280 nm).

In some embodiments, emitters are arranged along the inner sidewalls, upper rim, or submerged base of the tub, either in fixed panels or as directional arrays. In embodiments requiring submersion, emitters are housed in IP68-rated (or equivalent) waterproof panels or transparent waterproof enclosed casings that may be dropped into the tub. Emitter arrays may also be mounted on or behind transparent exterior panels.

In some embodiments, each emitter or emitter group is individually addressable and may be modulated in terms of wavelength, intensity, duty cycle, and pulsing pattern. Power output per wavelength is independently controlled to compensate for the differential attenuation of each wavelength in the liquid, ensuring adequate irradiance at the user's skin surface and tissue.

In some embodiments, red light in the range of approximately 630-670 nm stimulates mitochondrial cytochrome c oxidase activity, increases ATP production, and reduces inflammatory cytokines. NIR light (approximately 810-850 nm) penetrates deeper into muscular and subcutaneous tissue and promotes vasodilation through endothelial nitric oxide release. NIR light further supports post-immersion thermal homeostasis by accelerating tissue rewarming and improving circulation to hypoperfused extremities. Blue light stimulates alertness via melanopsin activation and may assist in microbial load reduction in shared-use environments. Green light may activate parasympathetic responses and reduce anxiety. Research indicates that lowering tissue temperature may enhance certain photobiomodulation effects at the molecular and cellular level. Prior art has addressed sequential use of cold therapy and light therapy (contrast therapy); the present invention is distinguished by the simultaneous delivery of cold liquid immersion and multi-wavelength photobiomodulation, which produces synergistic effects at the cellular and molecular level not achievable by sequential application alone.

In some embodiments, the light therapy subsystem may include timer-controlled LED color transitions across wavelengths, user-defined or default time intervals and power levels, and session completion indicators. A mobile application (via Wi-Fi, Bluetooth, LTE, or any wireless technology) may enable remote configuration of light intensity, duration, emitter color transitions, and therapy modes. The system supports storage of user profiles and therapy presets, allowing personalized light therapy parameters to be saved and recalled across sessions.

### V. Sound Therapy Subsystem

In some embodiments, the system further includes an underliquid acoustic therapy module comprising one or more transducers capable of producing sub-audible, audible, and ultrasonic frequencies. Transducers may be mounted in the walls or base of the tub and are configured to transmit vibrational energy directly into the liquid column. Frequencies may range from approximately 20 Hz to 200 Hz for low-frequency whole-body vibration, through mid-range auditory neuromodulation frequencies, to over 100 kHz for ultrasonic applications.

In some embodiments, specialized audio profiles may be used to promote relaxation, enhance focus, or support breath synchronization via brainwave entrainment protocols, such as delta (1-4 Hz), theta (4-8 Hz), or alpha (8-12 Hz) binaural beats.

In some embodiments, low-frequency acoustic energy introduced into the tub liquid may also induce microconvection, improving temperature uniformity and enhancing the rate of thermal exchange between the user's skin and the surrounding liquid. Ultrasonic or targeted vibrational profiles may be used in conjunction with mechanical actuators to prevent ice adhesion and facilitate ice dislodgement from tub walls and cooling coil surfaces.

### VI. Mobility System

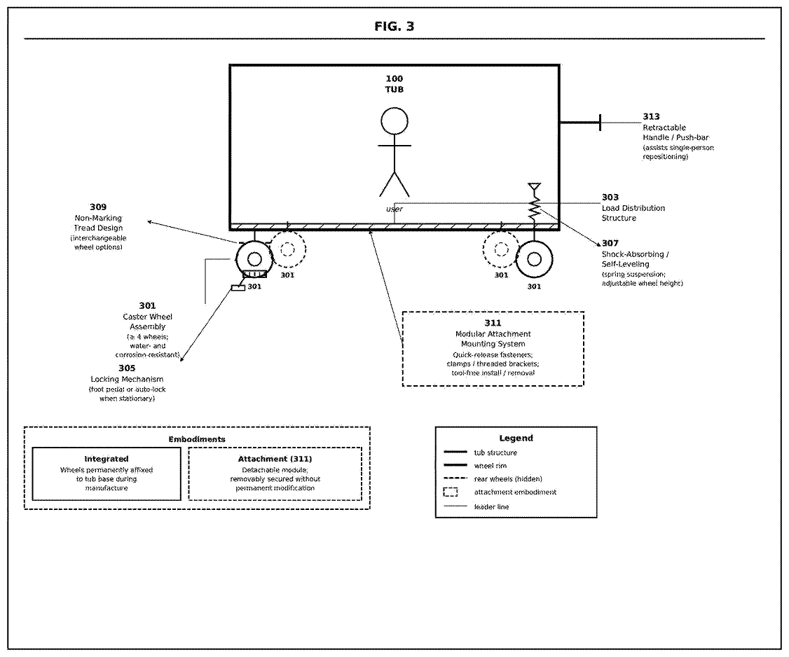

FIG. 3 is a schematic diagram of an embodiment of the caster wheel mobility system, illustrating the caster wheel assembly (301), load-distribution and weight-bearing structure (303), locking mechanism (305), shock-absorbing and self-leveling adjustments (307), non-marking tread design (309), modular attachment mounting system (311), and smooth maneuverability features (313).

In some embodiments, the cold plunge tub is provided with a caster wheel assembly configured to facilitate transportation and repositioning without heavy lifting. The assembly comprises a set of wheels, a structural mounting assembly, and a locking mechanism. Wheels are constructed from corrosion-resistant materials such as reinforced rubber, polyurethane, or stainless steel.

In some embodiments, in the integrated embodiment, caster wheels are permanently affixed to the underside of the tub, forming part of the tub's base structure, secured within reinforced mounting brackets molded into or bolted onto a reinforced structural plate. At least four caster wheels are positioned at or near the corners of the tub base to ensure balanced weight distribution. In the attachment embodiment, the caster wheel system is a detachable module that is removably secured to an existing tub using adjustable mounting brackets, clamp-based fasteners, threaded bolts, or quick-release latches, enabling tool-free installation and removal without permanent modification to the tub. The attachment unit may include an extendable handle or lifting mechanism. In certain embodiments, interchangeable wheel options allow users to select caster types suited to the operating environment, such as smooth wheels for indoor floors and larger-diameter wheels for outdoor or rough surfaces.

In some embodiments, the locking mechanism allows the user to engage wheel brakes to prevent unintended movement when the tub is in use. Engagement may be manual via a foot pedal or, in certain configurations, automatic when the tub has been stationary for a predetermined period. An optional self-leveling mechanism adjusts individual wheel height to maintain a stable and balanced position on sloped or uneven flooring. An optional shock-absorbing suspension system reduces vibrational impact during transport. Wheels feature a non-marking tread design and may include weather-resistant coatings for outdoor use.

### VII. Control System

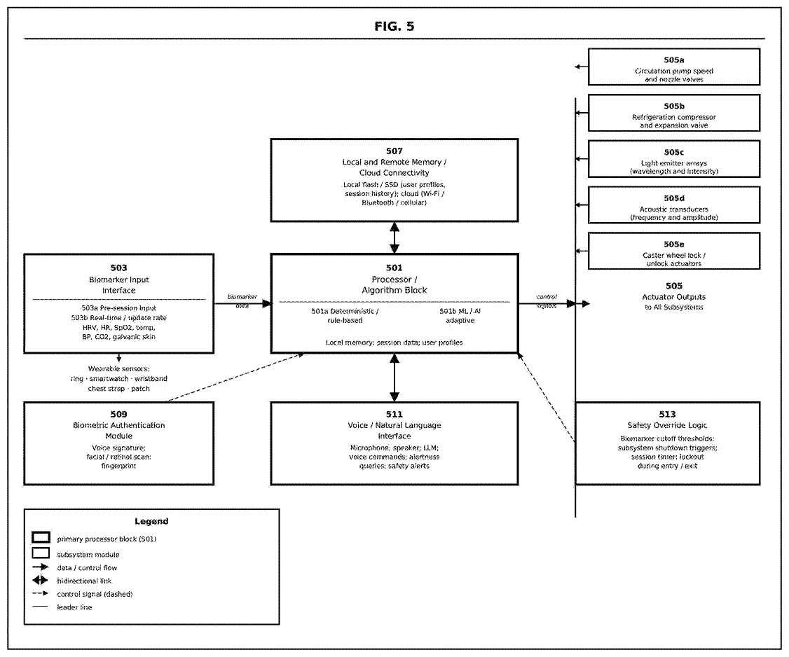

FIG. 5 is a schematic diagram of an embodiment of the intelligent control architecture, illustrating the processor and algorithm block (501), biomarker input interface (503), actuator outputs to all subsystems (505), local and remote memory/cloud connectivity (507), biometric authentication module (509), voice/natural language interface (511), and safety override logic (513).

In some embodiments, the system may be operated via a user-controlled interface that accepts direct user settings of liquid temperature, ice formation parameters, circulation rate and pattern, light therapy parameters, and sound therapy parameters. The interface may include mechanical knobs, digital input buttons, capacitive receptors, touchscreens, touchpads, remote controls, or voice commands.

In some embodiments, the controller incorporates a processor executing control algorithms that may be deterministic, rule-based, or dynamically adaptive. The controller is communicatively coupled to one or more biomarker data sources, including wearable sensors and physiological monitoring devices such as rings, wristbands, chest straps, smartwatches, physiological monitoring patches, or finger clips, each configured to collect physiological data from the user. In some embodiments, biomarker data may be collected by non-wearable sensors that do not require contact with or attachment to the user. Non-wearable biomarker sensing may include, without limitation, infrared cameras or thermal imagers configured to measure skin or body surface temperature remotely; optical sensors configured to detect blood oxygen saturation, CO2 levels, or respiratory rate through analysis of reflected or transmitted light; microphones or acoustic sensors configured to detect respiratory patterns, heart sounds, or vocal characteristics; eye-tracking cameras configured to monitor eye movement, blink rate, or pupillary response; and other remote sensing technologies capable of detecting physiological state without direct contact. Biomarker inputs may include, without limitation, core body temperature, skin surface temperature, heart rate, HRV, blood pressure, blood oxygen saturation (SpO2), respiratory rate, CO2 output, muscle activation, and localized vascular response.

In some embodiments, biomarker input may be pre-measured and provided to the controller prior to system operation, or streamed in real time during a therapy session, or both. The controller may update biomarker readings at a defined update rate during the session and dynamically adjust active system parameters in response to physiological changes. For example, a decrease in peripheral blood circulation or increase in HRV may trigger the controller to reduce circulation intensity or raise liquid temperature to avoid a hypothermic response; conversely, if thermal and circulatory indicators remain within target ranges, the controller may increase circulator stimulation or prolong exposure duration.

In some embodiments, the controller is equipped with machine learning (ML) or artificial intelligence (AI) capabilities, including neural network and other adaptive algorithms, and may store previous control actions and user-specific data in local or remote (cloud) memory, accessible through secure authentication, which may include voice signature, facial recognition, retinal scan, or fingerprint scan. The controller may operate a learning algorithm that accumulates session data and session history including previous control actions and associated biomarker values, and refines future session parameters autonomously over time using historical user outcomes.

In some embodiments, the controller may include a voice or natural language interface (AI language model or equivalent) for user identification, communication of therapeutic goals, override of specific biomarker inputs, and delivery of safety information and alerts. The controller may prompt the user with audible queries to verify user alertness and detect early signs of physiological vulnerability or a vulnerable physiological state.

In some embodiments, the controller is configured with a secure cloud-connected portal enabling remote configuration by licensed healthcare providers or clinicians, who may create and transmit user-specific therapy plans authenticated at the time of system use via secure password or biometric identification. In an AI-enabled cloud embodiment, a centralized platform may provide cloud-based coordination of multiple health maintenance devices (cold plunge, sauna, massage chair, cardiac monitor) for a single user or across multiple users, managed through a unified control interface. Cloud-based access, features, and strategic planning may be subscription-based.

In some embodiments, sensors within the system may measure liquid temperature, degree of ice formation, flow rates, salinity, liquid-borne vibration, acoustic signals, ultrasonic resonance, solid-state ice content, and other parameters, providing feedback to the controller for dynamic adjustment of all subsystem settings.

In some embodiments, the system may include safety features including session timers, LED or audible alerts when liquid has reached a target temperature or a cycle is complete, basic safety lockout protocols preventing unintentional subsystem activation during user entry or exit, biomarker-based threshold cutoffs triggering safety overrides, and safety alerts delivered via visual display, app notifications, and/or audible cues. All electronic components in contact with or proximate to liquid are waterproofed, and the system includes GFCI protection, circuit breakers, and electrical grounding.

In some embodiments, when integrated under centralized AI control, the light therapy, sound therapy, hydro-circulator, and ice-forming subsystems may operate in tandem to deliver a multi-modal, sensor-driven, and fully adaptive therapeutic experience. When a common biomarker condition is detected across subsystems, the controller may respond to that condition by simultaneously adjusting multiple subsystems. For example, upon detection of a rapid drop in peripheral temperature combined with elevated HRV, the controller may simultaneously reduce ice production, initiate red/NIR light delivery, and play alpha-range acoustic profiles to stabilize autonomic tone while maintaining immersion.

In some embodiments, system diagnostics verify the operational state of all active components, including emitter temperature and electrical function of light-emitting elements, acoustic resonance levels of transducers, and refrigerant pressure within the cooling circuit. Biomarker cutoff thresholds defined for each user profile trigger safety overrides when monitored parameters exceed defined limits. The integrated simultaneous use of cold immersion with light and sound therapy enables therapeutic protocols applicable to neurorehabilitation, pain management, cognitive reset, and integrative performance recovery, expanding the utility of the system beyond conventional cold plunge applications.

### VIII. Water Sanitation

In some embodiments, the system may include one or more stages of water sanitation, including without limitation: ozone injection, ultraviolet sterilization, micron-level particulate filtration, paper or traditional pool filtration, and any combination thereof. Filter replacement alerts may be generated in response to flow monitoring (detecting reduction in throughput) and/or time-based logic, delivered via visual display, app notification, and/or audible cue.

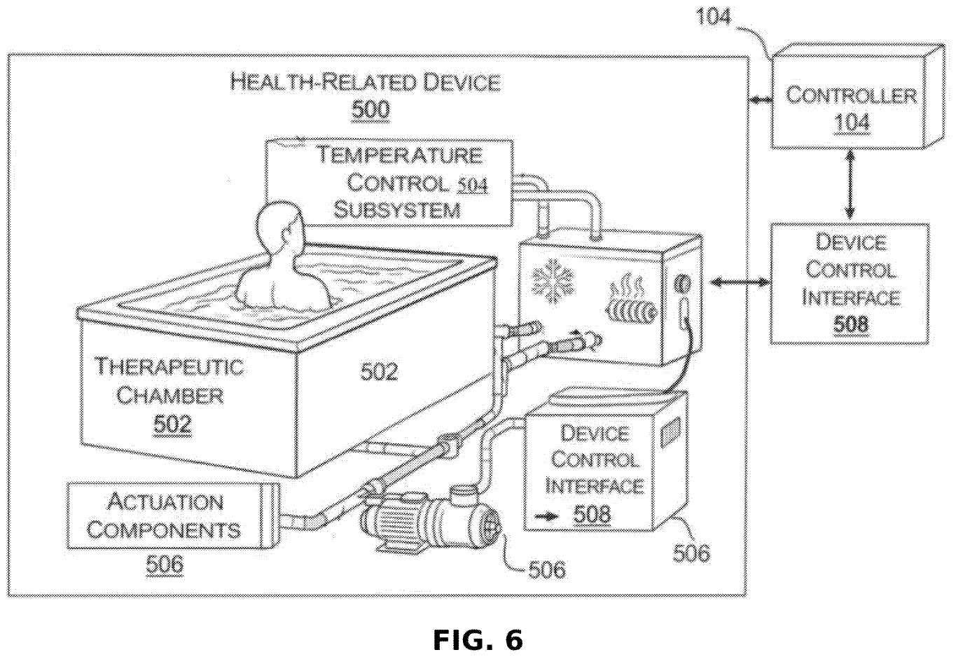

### IX. Cold Plunge System Overview (FIG. 6)

FIG. 6 is an illustration of an embodiment of the cold plunge therapy system (100) in assembled form, showing the therapeutic chamber (502) with a user immersed, the temperature control subsystem (504), actuation components including the circulation pump (506), the device control interface (508), and the controller (104) connected externally. This figure illustrates the physical integration of the tub, cooling, circulation, and control subsystems as a complete health-related device.

FIG. 6 illustrates the cold plunge therapy system (100) in an assembled, operational configuration. The therapeutic chamber (502) comprises the tub structure configured to retain liquid and receive an immersed user. The temperature control subsystem (504) is operatively coupled to the therapeutic chamber and comprises the refrigeration circuit, cooling coils, and associated controls described in Section III, configured to reduce and maintain liquid temperature including to sub-freezing levels with ice formation.

In some embodiments, the actuation components (506) comprise the circulation pump and associated plumbing that draws liquid from the therapeutic chamber and returns it via the circulator nozzles described in Section II, as well as any additional actuators for the cooling, light therapy, and sound therapy subsystems. The device control interface (508) is communicatively coupled to the actuation components and provides the local user-facing control panel through which operational parameters are set and monitored. The controller (104) is connected externally to the device control interface and executes the control algorithms described in Section VII, receiving biomarker inputs and issuing control signals to all subsystems.

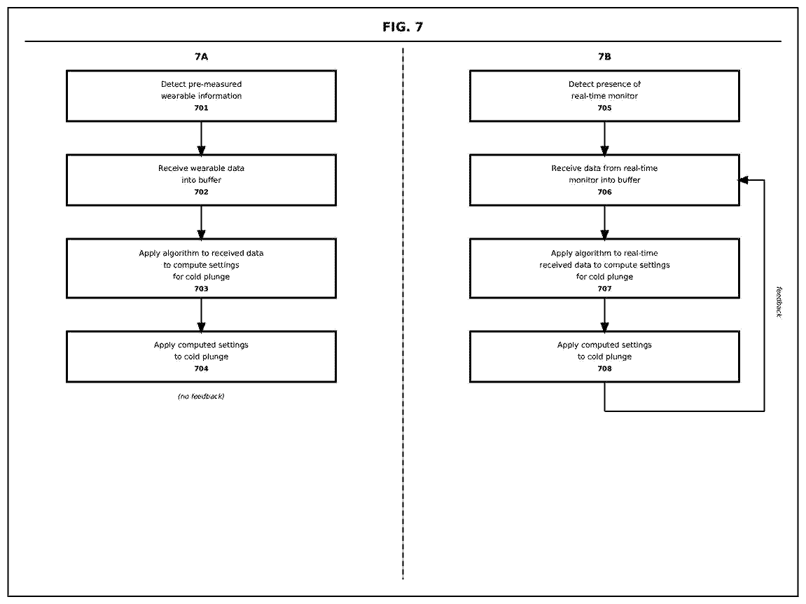

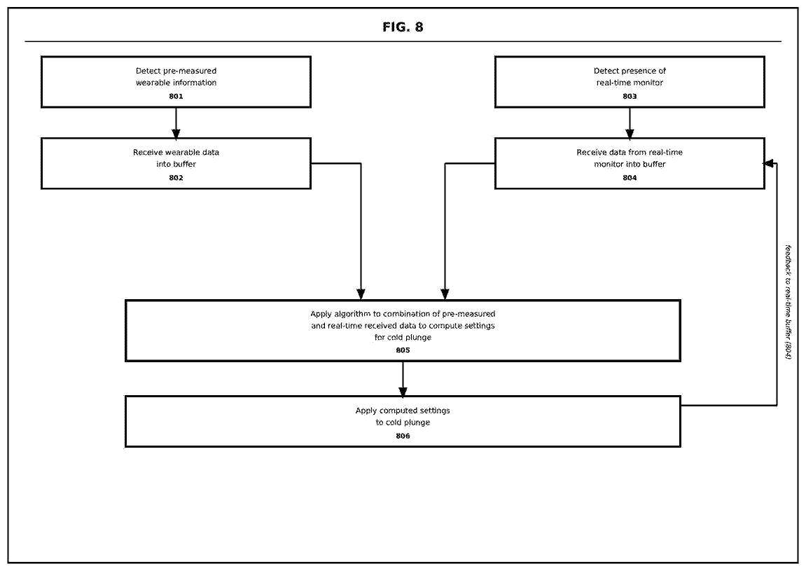

### X. Biomarker-Based Control Embodiments (FIGS. 7 and 8)

FIG. 7 is a block diagram showing an embodiment in which pre-measured biomarker information and real-time biomarker information can be used independently to control the cold plunge. FIG. 7 contains two block diagrams, 7A and 7B. In FIG. 7A, block 701 detects pre-measured wearable information. Block 702 receives the wearable data into a buffer. Block 703 applies an algorithm to the received data to compute settings for the cold plunge. Block 704 applies the computed settings to the cold plunge. There is no feedback path in FIG. 7A; the pre-measured biomarker values are used once to set the cold plunge parameters before the session begins. In FIG. 7B, block 705 detects the presence of a real-time monitor. Block 706 receives data from the real-time monitor into a buffer. Block 707 applies an algorithm to the real-time received data to compute settings for the cold plunge. Block 708 applies the computed settings to the cold plunge. A feedback path returns from block 708 back to block 706, so that the real-time monitor data is continuously received and the settings are continuously updated during the session.

FIG. 8 is a block diagram showing an embodiment in which pre-measured biomarker information and real-time biomarker information can be used together to control the cold plunge. Two input paths feed into a common processing block. In the first path, block 801 detects pre-measured wearable information, and block 802 receives the wearable data into a buffer. In the second path, block 803 detects the presence of a real-time monitor, and block 804 receives data from the real-time monitor into a buffer. Both paths converge into block 805, which applies an algorithm to the combination of pre-measured and real-time received data to compute settings for the cold plunge. Block 806 applies the computed settings to the cold plunge. A feedback path returns from block 806 back to block 804, so that the real-time monitor data is continuously received into the buffer and the combined algorithm continuously updates the cold plunge settings throughout the session.

What is claimed is:

1. A cold plunge therapy system comprising: (a) a tub configured to retain liquid; (b) a hydro-circulation subsystem comprising at least one intake port configured to draw liquid from the tub interior into the pump system, at least one pump, and at least one nozzle configured to expel liquid from the pump system into the tub interior; (c) an ice-forming cooling subsystem comprising a set of one or more thermally conductive coils coupled to a refrigeration circuit configured to reduce liquid temperature in the tub and promote formation of ice within the tub; and (d) a control system configured to receive user inputs and operate the hydro-circulation subsystem and the ice-forming cooling subsystem in accordance with one or more operational parameters.

2. The system of claim 1, further comprising a light therapy subsystem comprising an array of light-emitting elements housed in waterproof panels or enclosures, configured to emit at least two wavelengths selected from the group consisting of: (a) infrared in the range of 750 nm-1 mm; (b) red light in the range of 620-750 nm; (c) green light in the range of 500-570 nm; (d) blue light in the range of 410-480 nm; and (e) ultraviolet light; wherein the control system is configured to activate and modulate the light therapy subsystem during a cold plunge session, and wherein the control system independently controls power output per wavelength to compensate for differential attenuation of each wavelength in the liquid.

3. The system of claim 1, further comprising a sound therapy subsystem comprising one or more liquid-coupled transducers configured to emit acoustic energy at sub-audible, audible, or ultrasonic frequencies, wherein the transducers are mounted to the tub walls or base and are configured to transmit vibrational energy into the liquid, and wherein the sound therapy subsystem is configured to emit waveforms corresponding to neural entrainment frequencies to promote parasympathetic activation.

4. The system of claim 3, wherein the sound therapy subsystem is further configured to operate at ultrasonic frequencies to dislodge ice from the interior surfaces of the tub and the thermally conductive coils.

5. The system of claim 1, further comprising a caster wheel assembly coupled to the tub, the assembly comprising: (a) a plurality of caster wheels constructed from corrosion-resistant material; (b) a locking mechanism configured to selectively secure the tub in a stationary position; and (c) a mounting assembly.

6. The system of claim 5, wherein the caster wheel assembly is configured in a first configuration in which the wheels are permanently affixed to the tub base, or in a second configuration in which the wheels are removably secured to an existing tub without permanent modification; and wherein the caster wheel assembly further comprises a shock-absorbing suspension system and a self-leveling mechanism configured to adjust individual wheel height to compensate for uneven surfaces.

7. The system of claim 1, wherein the hydro-circulation subsystem comprises a plurality of pumps arranged in a series configuration, wherein one or more pumps are independently controllable in speed and output aperture.

8. The system of claim 1, wherein the hydro-circulation subsystem comprises a plurality of pumps arranged in a parallel configuration, wherein one or more pumps are independently controllable in speed and output aperture.

9. The system of claim 8, wherein the plurality of pumps in the parallel configuration each pass liquid through separate filtration or sanitation pathways, wherein at least one pathway comprises a particulate filter and at least one other pathway comprises an ozone or ultraviolet sanitation unit.

10. The system of claim 1, wherein one or more intake ports, one or more nozzles, or both, are adjustable in direction to control liquid flow patterns within the tub, and wherein the nozzles may be arranged in discrete anatomical zones with each nozzle independently adjustable in direction, flow rate, and pulsing pattern to deliver hydrodynamic stimulation.

11. The system of claim 1, further comprising one or more nozzles configured to deliver warmed or ambient-temperature liquid to a localized region of the user's body to prevent cooling of that region, wherein isolation of the region is achieved by one or more of: blocking a portion of the tub from the main liquid flow; enclosing the region with a wrap; or providing a wrap with inlet and outlet tubes.

12. The system of claim 1, wherein the ice-forming cooling subsystem further comprises an ice-shedding mechanism configured to dislodge ice formed on the tub interior surfaces, the ice-shedding mechanism comprising at least one of: a mechanical vibrator coupled to the tub; a sonic or ultrasonic transducer; or liquid circulation forces generated by the hydro-circulation subsystem.

13. The system of claim 1, wherein the ice-forming cooling subsystem further comprises an automatic de-icing mode in which the control system periodically increases the coil temperature to prevent ice accumulation, the de-icing mode being triggered by one or more of: a thermal sensor detecting ice thickness exceeding a threshold; a preprogrammed time interval; or a manual command.

14. The system of claim 1, wherein the control system comprises a controller comprising: (a) a processor configured to execute a control algorithm; (b) a biomarker input interface configured to receive physiological data from at least one sensor prior to a therapy session, during a therapy session, or both; and (c) control logic configured to adjust operational parameters of the hydro-circulation subsystem, the ice-forming cooling subsystem, and any additional subsystems present, in response to the received biomarker data.

15. The system of claim 14, wherein the biomarker data comprises at least one of: core body temperature, skin surface temperature, heart rate, heart rate variability (HRV), blood pressure, blood oxygen saturation (SpO2), respiratory rate, CO2 output, galvanic skin response, or muscle activation level.

16. The system of claim 14, wherein the biomarker input interface is configured to receive data from a wearable device selected from the group consisting of a ring, a smartwatch, a wristband, a chest sensor, a physiological monitoring patch, and a finger clip.

17. The system of claim 14, wherein the processor executes a machine learning or artificial intelligence algorithm, and wherein the controller stores session data in local or remote memory to refine future session parameters using prior user outcomes.

18. The system of claim 14, wherein the controller further comprises a biometric authentication module configured to validate user identity using at least one of: voice signature, facial recognition, retinal scan, or fingerprint scan; and wherein the controller stores user-specific therapy profiles accessible only upon successful authentication.

19. The system of claim 14, wherein the controller further comprises a voice or natural language interface configured to: receive user commands and therapeutic goal inputs; present audible queries to the user to assess alertness and detect early signs of physiological vulnerability; and issue audible safety alerts or override commands.

20. The system of claim 14, wherein the controller is further configured to communicate with a remote healthcare provider portal to receive user-specific therapy plans, wherein execution of such plans is conditioned upon authentication of the user at the time of system use.

21. The system of claim 14, wherein the controller further comprises a cloud-based coordination module configured to synchronize therapy protocols across a plurality of health maintenance devices, including at least one other therapeutic device, in accordance with biomarker feedback or clinician-defined rules.

22. The system of claim 1, further comprising one or more sensors configured to measure at least one of: liquid temperature, degree of ice formation, liquid flow rate, salinity, liquid-borne vibration, acoustic resonance, or solid-state ice content within the tub, wherein measurement data is provided to the control system as a feedback input for adjustment of system parameters.

23. The system of claim 1, further comprising a water sanitation subsystem comprising at least one of: ozone injection, ultraviolet sterilization, or particulate filtration; and a filter replacement alert system triggered by one or more of flow-reduction detection or time-based usage monitoring.

24. A cold plunge therapy system comprising: (a) a tub configured to retain liquid; (b) a cooling subsystem comprising a liquid chiller configured to reduce liquid temperature, and an ice-forming mechanism configured to introduce ice into the liquid to further reduce the liquid temperature; (c) a hydro-circulation subsystem comprising at least one intake port configured to draw liquid from the tub interior into the pump system, at least one pump, and at least one nozzle configured to expel liquid from the pump system into the tub interior; and (d) a controller comprising a processor and a biomarker input interface configured to receive physiological data from at least one sensor, the controller configured to adjust at least liquid temperature and circulation intensity in response to the received physiological data.

25. The system of claim 24, wherein the control system is configured to modulate the wavelength, intensity, duty cycle, and pulsing pattern of the light therapy subsystem in response to biomarker data received from at least one sensor; and wherein, when a sound therapy subsystem is present, the control system is further configured to coordinate operation of both the light therapy subsystem and the sound therapy subsystem in response to a common biomarker condition to maintain a user-specific therapeutic profile.

26. A method of delivering cold plunge therapy comprising: (a) filling a tub with liquid; (b) chilling the liquid using a refrigeration circuit coupled to the tub via coils; (c) forming ice within the tub; (d) circulating the liquid using at least one pump and a plurality of nozzles to deliver hydrodynamic stimulation; and (e) adjusting at least one of liquid temperature, ice formation rate, or circulation intensity.

27. The method of claim 26, further comprising emitting electromagnetic radiation at two or more wavelengths into the liquid and toward the immersed user using light-emitting elements housed in waterproof enclosures, wherein the wavelengths are selected from infrared, red, green, blue, and ultraviolet, and wherein power output is independently controlled per wavelength.

28. The method of claim 26, further comprising transmitting acoustic energy into the liquid using liquid-coupled transducers at sub-audible, audible, or ultrasonic frequencies, to induce at least one of: somatosensory vibration; brainwave entrainment; acoustic microconvection within the liquid; or ice dislodgement from tub interior surfaces.

29. The method of claim 26, wherein adjusting operational parameters is performed by a processor executing a machine learning or artificial intelligence algorithm that receives biomarker data in real time, stores session history, and autonomously modifies future session parameters using prior user outcomes.
